SEQUENCE LISTING (4 sequences)

SEQ ID NO: 1 - DNA, 22 bp, Artificial Sequence
  Primer
tgctcaaagaagtgcggaccct

SEQ ID NO: 2 - DNA, 21 bp, Artificial Sequence
  Primer
gccccactgcttctccttcct

SEQ ID NO: 3 - DNA, 20 bp, Artificial Sequence
  Primer
tttggggcatggacattgac

SEQ ID NO: 4 - DNA, 20 bp, Artificial Sequence
  Primer
cccaccttatgagtccaagg

(19) United States
(12) Patent Application Publication
(10) Pub. No.: US 2005/0042226 A1
(43) Pub. Date: Feb. 24, 2005

(54) Treatment of chronic human viral hepatitis

(75) Inventors: Gary Levy, Thornhill (CA); Philip Marsden, Toronto (CA)

Correspondence Address: BERESKIN AND PARR, SCOTIA PLAZA, 40 KING STREET WEST-SUITE 4000 BOX 401, TORONTO, ON, M5H 3Y2, CA

(73) Assignee: Trillium Therapeutics Inc., Toronto (CA)

(21) Appl. No.: 10/875,528

(22) Filed: Jun. 25, 2004

Related U.S. Application Data
(60) Provisional application No. 60/482,448, filed on Jun. 26, 2003.

(51) Int. Cl. C12Q 1/68; A61K 39/42

(52) U.S. Cl. 424/161.1

(57) ABSTRACT

The use of Fgl2 inhibitors to treat and control the progression of human Hepatitis B virus-induced hepatitis is described. Inhibitors of Fgl2 include antibodies to Fgl2.

This application claims the benefit under 35 USC §119(e) from U.S. provisional patent application Ser. No. 60/482,448, filed Jun. 26, 2003, which is incorporated herein by reference in its entirety.

### FIELD OF THE INVENTION

This invention relates to fibroleukin, a protein also known as Fgl2, which is implicated in immune coagulation. More particularly, the invention relates to the use of Fgl2 inhibitors to treat, and to control the progression of, human Hepatitis B virus-induced hepatitis.

### BACKGROUND TO THE INVENTION

Many of the viral pathogens known to cause liver disease are not directly cytopathic for the hepatocyte. For instance, the acute and chronic liver diseases caused by the hepatitis B virus reflect hepatocellular injury believed to be induced by an immune response to the virus rather than by direct viral-induced hepatocellular necrosis. For example, interferon gamma derived from activated T-cells within the liver figures prominently in viral-induced liver disease in humans as well as in animal models (1). Another especially important effector phase in the immune response to viral infection of the liver is fibrin deposition and thrombosis within the microvasculature (2).

Tissue factor (TF), the transmembrane receptor for Factor VII, is the major procoagulant for the classical pathway and is known to initiate the extrinsic pathway of coagulation in response to lipopolysaccharide (LPS) (3, 4). In the presence of calcium, the TF/Factor VIIa complex activates the zymogen precursor Factor X. Factor Xa is a serine protease that is the classically known physiological activator of prothrombin. Efficient thrombin generation by Factor Xa requires calcium, an electronegative phospholipid surface, and the nonenzymatic cofactor, factor Va. Current evidence indicates that the pathways by which vascular thrombosis are elicited in viral hepatitis are mechanistically distinct from the classical pathways of coagulation induced by mechanical trauma or endotoxin (LPS). The in vitro induction of a procoagulant response in mononuclear cells by murine hepatitis virus type 3 (MHV-3) correlates with the disease susceptibility in strains of mice (5). MHV-3 is a member of the Coronoviridae, a group of positive-stranded, enveloped RNA viruses. Peritoneal macrophages from BALB/c or C57Bl/6 mice, strains in which MHV-3 infection results in fatal acute fulminant hepatitis, respond to the virus with a robust procoagulant response. In contrast, peritoneal macrophages from A/J mice, a strain fully resistant to MHV-3, generate no increase in procoagulant above control levels. This procoagulant response represented a functional marker for susceptibility to MHV-3 infection in inbred strains of mice suggesting that this response is important in the pathogenesis of coronavirus MHV-3-induced disease.

Activated endothelial cells and macrophages express distinct cell-surface procoagulants important for both the initiation and localization of fibrin deposition in viral-induced liver disease. The cloning and characterization of a novel procoagulant induced by MHV-3 infection of murine macrophages has been reported (6). Fgl2/fibroleukin is an immune coagulant with the ability to directly cleave prothrombin to thrombin (6-10). Initially cloned from CD8⁺ cytotoxic T cells, Fgl2/fibroleukin protein shares homology of its carboxyl terminus with fibrinogen β and γ chains (11). Fgl2/fibroleukin protein generated in a baculovirus expression system exhibits no measurable prothrombinase activity until reconstituted into negatively charged phospholipids vesicles (e.g. phosphatidyl-L-serine containing) (10). As with Factor Xa, catalytic activity is dependent on calcium and is enhanced by factor Va. Site-directed mutagenesis of Ser89 of the murine protein indicated that Fgl2/fibroleukin has the characteristics of a serine protease and that Ser89 was critical for the prothrombinase activity of Fgl2. In contrast to Factor Xa, the Fgl2/fibroleukin prothrombinase activity is not inhibited by antithrombin III.

The human and murine genes for the Fgl2/fibroleukin prothrombinase have been cloned and characterized and localized to chromosomes 7 and 5, respectively (8, 12). Of note, expression of the Fgl2/fibroleukin prothrombinase mRNA is subject to transcriptional regulation, exhibiting both constitutive and inducible expression. For example, steady-state mRNA expression of Fgl2/fibroleukin prothrombinase is markedly upregulated by interferon-gamma (13, 14). In vivo studies indicate robust expression of the Fgl2/fibroleukin prothrombinase in hepatic macrophages and endothelial cells in MHV-3 murine hepatitis models and patients with fulminant hepatic failure (7, 15).

Given that the Fgl2/fibroleukin may be important for both the initiation and localization of fibrin deposition in viral hepatitis the inventors generated an Fgl2/fibroleukin-deficient mouse. It has been reported that fibrin deposition and liver necrosis were markedly reduced and survival was increased in mice infected with the murine hepatitis virus strain 3 (MHV-3) Coronavirus. It remains to be determined whether, however, there is any nexus between the human conditions that result from infection with human hepatitis virus, and the condition of fulminant hepatitis that results in mice infected with the MHV-3 Coronavirus.

### SUMMARY OF THE INVENTION

It has now been determined that Fgl2 is upregulated in subjects afflicted with hepatitis B-virus induced disease, and particularly in patients with marked, chronic hepatitis B virus-induced disease. In accordance with the present invention, there is provided a method for treating patients afflicted with non-fulminant hepatitis, and more particularly patients afflicted with chronic viral hepatitis B, by administering an inhibitor of Fgl2, preferably to arrest the progression of said disease. In embodiments of the present invention, the Fgl2 inhibitor is administered to patients afflicted with marked, chronic viral hepatitis B. In other embodiments, the Fgl2 inhibitor is administered to patients afflicted with minimal, chronic viral hepatitis B, to inhibit the onset of marked chronic viral hepatitis B.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention and its preferred embodiments are now described in greater detail with reference to the accompanying drawings, in which:

FIG. 1 shows the generation of Fgl2/fibroleukin-deficient mice. (a) Targeting strategy for Fgl2 gene. LacZ reporter and PGK-neo gene was inserted within the first of two coding exons and the Fgl2/fibroleukin initiator methionine codon in exon 1 was mutated to a BamHI site. Arrowheads represent primers used for multiplex PCR genotyping. Probe used for genomic Southern blot analysis indicated by the dotted line. (b) Genomic Southern blot analysis of ES cell clones. 10.2 kb or 5.2 kb BamHI restriction fragments are evident in wild-type or knockout alleles, respectively. (c) Multiplex PCR for genotyping mice amplified a 319 bp-amplicon from the knockout allele or a 373 bp-amplicon from the wild-type allele. (d) RT-PCR analysis of MHV3-treated macrophages (8 hr). Neither constitutive nor induced mRNA was detected in the null knockout mice.

FIG. 2 shows that Fgl2/fibroleukin-deficient mice have hemorrhage at the feto-maternal interface. Zygotes and uterine tissue were examined by light microscopy and immunohistochemistry using a polyclonal anti-Fgl2 antibody. Feto-maternal interface of Fgl2/fibroleukin^{+/+} zygotes at day 8.5 pc as assessed by haematoxylin eosin staining (a) and Fgl2 immunostaining (b). Murine trophoectoderm is positive for Fgl2 immunostaining. Fgl2/fibroleukin^{-/-} zygotes showed moderate to severe haemorrhage at implantation sites with extravasation of maternal erythrocytes (rbc, red blood cells, arrowhead) between the mural trophoectoderm and Reichert's membrane (arrow) at the anti-mesometrial pole as assessed by haematoxylin and eosin staining (c) and negative Fgl2 immunostaining (d)(see Methods).

FIG. 3 shows mortality in Fgl2/fibroleukin deficient mice. (a) Mortality in MHV-3 infected (1000 pfu) Fgl2/fibroleukin^{-/-} mice was significantly blunted compared to Fgl2/fibroleukin^{+/+} littermates, with heterozygotes showing an intermediate phenotype (n=10 per group). C57Bl/6 and A/J strains represent susceptible and resistant strains, as previously reported. (b) Mortality is not affected in LPS-treated Fgl2/fibroleukin deficient mice. The C3H HeJ strain is known to be LPS-hyporesponsive.

FIG. 4 shows fibrin deposition and cellular necrosis in livers of Fgl2/fibroleukin-deficient mice following MHV-3 infection. (a) Liver from C57Bl/6 (susceptible) mice 3 d post-infection. The inventors note widespread confluent fibrin deposition and necrosis involving greater than 90% of liver tissue. (b) Liver from A/J (resistant) mice. Liver tissue is normal with no fibrin deposition or necrosis. (c) Liver from Fgl2/fibroleukin^{+/+} mouse. The inventors noted that the liver shows confluent fibrin deposition and necrosis. (d) Liver from Fgl2/fibroleukin^{+/-} mouse. Fibrin deposition and necrosis and scattered foci are seen. (e) Liver from Fgl2/fibroleukin^{-/-} mouse. The inventors noted that the livers were essentially free of fibrin deposition and necrosis. Only small rare foci of fibrin deposition and necrosis were evident.

FIG. 5 shows Fgl2/fibroleukin expression and fibrin deposition in human viral hepatitis B. Liver biopsy from a patient with marked chronic hepatitis B: (a) Area of necrosis and collapse of parenchyma is shown. The few remaining hepatocytes in this field show ballooning degeneration. Hematoxylin and eosin stain, ×400. (b) Area of necrosis and collapse on left. Many macrophages are present in the area of collapse as shown by the positive CD68 immunoperoxidase stain, ×400. (c) Area similar to that shown in (B) showing positivity of Fgl2/fibroleukin by in-situ hybridization in the same distribution of the CD68 positive macrophages, ×200. (d) Another area of acute necrosis and collapse showing positivity using Fgl2/fibroleukin antibody by immunoperoxidase stain, ×400. (e) Similar area of necrosis to that shown in (d), fibrin deposits by immunoperoxidase staining are shown by arrows, ×400. (f) Patient with minimal chronic hepatitis B. This micrograph is representative and shows no evidence of hepatocellular necrosis, but a mixed mild inflammatory infiltrate confined to the portal and septal connective tissue. There is no evidence of Fgl2/fibroleukin in the parenchyma or in the areas of fibrosis areas by immunoperoxidase staining for Fgl2/fibroleukin, ×200.

### DETAILED DESCRIPTION OF THE INVENTION

Chronic infection with hepatitis B virus (HBV) affects an estimated 300 million persons on a global basis (24, 25). An estimated 1.25 million people in the United States are chronic carriers, defined as positive for hepatitis B surface antigen (HBsAg) for more than at least three months, e.g., generally more than about six months (26). Although most carriers do not develop hepatic complications from chronic hepatitis B, 15 to 40 percent will develop serious sequelae during their lifetime.

HBV-induced hepatitis is a condition that is distinct from fulminant viral hepatitis. Fulminant hepatitis is a condition that manifests only rarely in humans, in less than about 0.01% of the hepatitis population. It has both non-viral (e.g. drug) and viral causes. Fulminant viral hepatitis can be caused by a number of different agents, such as HepA and HepB, as well as the non-hepatitis ABCDE viruses. The condition thus is classified more with reference to the physiological conditions relevant to the failing liver, than with particular reference to the causative virus.

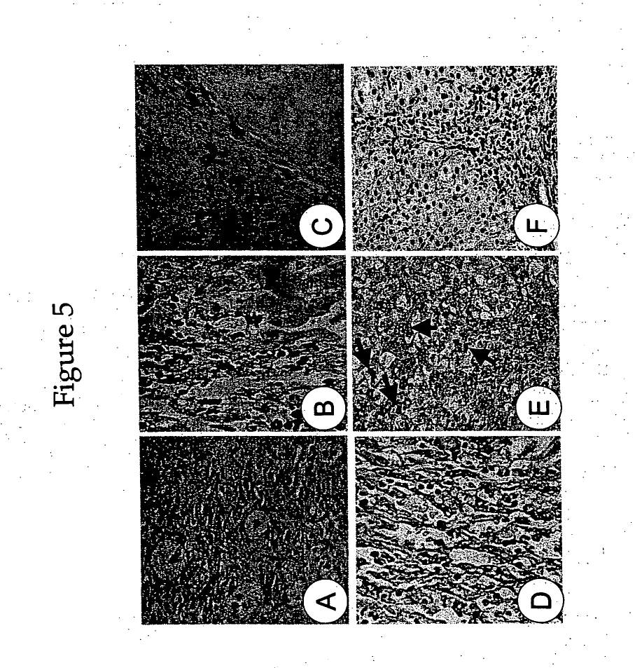

Viral hepatitis B is more particularly a condition that results from infection by the hepatitis B virus. Chronic viral hepatitis B is a condition other than fulminant hepatitis. Many chronic Hep B patients remain asymptomatic, while others may have only mild disease. Some, however, will develop cirrhosis and liver damage, which can ultimately lead to organ failure and death. Fgl2, by virtue of its procoagulant activity, may contribute to fibrin deposition within the liver and thus cause tissue damage. Fulminant viral hepatitis is a much more rapidly progressing disease characterized by the sudden onset of severe liver failure and hepatic encephalopathy, which can lead to coma and death. Patients presenting with chronic viral hepatitis B are those HBV-infected patients who are positive for HBsAg for more than about three to six months, when assessed using the conventional diagnostic test for HbsAg. This test is typically in the form of an ELISA test which detects the presence of the Hep B virus surface antigen in blood. As noted in the examples and Table 2 herein, 100% of all the patients in the present study (both minimal and chronic) were tested and confirmed positive (see Table 2). Within this patient population, those presenting with “marked” chronic viral hepatitis B are characterized by elevations in both the level of Fgl2 expression, and the level of fibrin deposition, relative to patients that present with “minimal” chronic viral hepatitis B (see Table 2, and FIG. 5).

Thus, the present invention relates to the treatment of subjects presenting with active HBV infection. In embodiments, the subjects are those presenting with chronic viral hepatitis B. In a particular embodiment, the subjects for treatment are those presenting with minimal chronic viral hepatitis B infection. In another particular embodiment, the subjects are those presenting with marked chronic viral hepatitis B infection.

In accordance with the present invention, patients presenting with active HBV infection are treated with an inhibitor of flg2. Accordingly, the present invention provides a method for treating a subject afflicted with chronic viral hepatitis B, comprising administering to said subject an inhibitor of Fgl2 in an amount effective to reduce Fgl2-mediated fibrin deposition.

The term “inhibitor of Fgl2” as used herein are those agents that inhibit Fgl2-mediated cleavage of prothrombin to thrombin. Agents having this property are identify readily using established in vitro assays of prothrombin cleavage, which detect either the accumulation of thrombin or the reduction of prothrombin in an incubation mixture that incorporates the agent together with labeled prothrombin and Fgl2 in enzymatically active form, i.e., Fgl2 in combination with calcium ion and phospholipid. Enzymatically active Fgl2 can, for instance, be obtained following its expression by a mammalian host cell such as CHO, as reported for instance by Levy et al in Am. J. Pathol., April 2000, 156(4):1217. It will be appreciated that the Fgl2 incorporated in the assay can be human Fgl2 having the sequence reported by Levy et al, infra, or an enzymatically active fragment thereof. Most desirably, the agent is one that inhibits the prothrombin cleavage mediated by wild type human Fgl2.

In one aspect of the present invention, the treatment of chronic viral hepatitis B is achieved by administering an antibody that binds to Fgl2. The Fgl2 antibody is one that inhibits the cleavage of prothrombin by Fgl2, and is administered to the viral hepatitis B subject using a dosing regimen that reduces the rate at which prothrombin is cleaved to yield thrombin. Most desirably, the subject is dosed with Fgl2 antibody in order to reduce the rate of fibrin deposition, thereby to reduce the severity of consequent damage to hepatocytes and the liver generally. Particularly for patients presenting with minimal chronic viral hepatitis B, the dosing regimen is aimed at maintaining the existing, relatively low levels of Fgl2 and fibrin deposition in such patients, and the dosing therefore is equivalent to the maintenance dosing in marked, chronic Hep B patients once the acute condition is under management. Accordingly, the present invention provides a method for treating a subject afflicted with chronic viral hepatitis B, comprising the step of administering to said subject an antibody to Fgl2 in an amount effective to reduce Fgl2-mediated fibrin deposition.

Specific dosing regimens useful in the present invention are revealed in appropriately designed clinical trials in human patients suffering from chronic viral hepatitis B. On the basis of results obtained in mice using antibody to murine Fgl2 (Li et al, 1992 and U.S. Pat. No. 6,403,089), it is anticipated that a useful dosing regimen in humans afflicted with marked chronic viral hepatitis B will generally entail the administration of Fgl2 antibody in doses that are the weight equivalent to 25-250 ug/mouse, on a dosing schedule that entails once or twice daily injection until significant Fgl2 activity subsides, with follow-on maintenance dosing at once or twice weekly intervals until significant symptoms subside. A resumption of dosing is desirable when HBV titres, and particularly when Fgl2 levels, return and subsist over prolonged periods.

Suitable therapies based on Fgl2 antibody are those which, in patients presenting with marked chronic viral hepatitis B, result in a reduction for instance in the level of active Fgl2, as determined using the prothrombinase assay herein described and established in the literature; and/or result in a reduction in the level or rate of fibrin deposition as determine using the fibrin detection assay also described herein and established in the literature.

For administration, the Fgl2 inhibitor is desirably formulated for administration by infusion or by injection, by any desired route include subcutaneous, intramuscular, intravenous or the like. The Fgl2 inhibitor is desirably formulated in any aqueous, physiologically tolerable vehicle, such as saline, and phosphate buffered saline, e.g., 0.9% PBS.

Suitable Fgl2 antibodies thus are those that neutralize or inhibit the prothrombinase activity of Fgl2. Suitable such antibodies include polyclonal antibodies, as well as monoclonal antibodies, and active fragments thereof. The antibodies can be non-human antibodies, but it is desirable in this case to generate the more tolerated versions thereof such as chimeric and humanized antibodies. Most desirably, the Fgl2 antibody is an antibody to human Fgl2.

The antibodies can be raised using, as antigen, the wild type Fgl2 protein, or any fragment or domain thereof that retains the epitope(s) involved in its cleavage interaction with prothrombin. Antibodies so raised can then be screened using the prothrombin cleavage assay noted above and established in the art, and antibodies that inhibit such cleavage can be selected for use. Suitable antibodies include, but are not limited to antibodies of the IgG2 isotype, such as IgG2a.

Antibodies that bind to Flg2 can be prepared using techniques known in the art. For example, by using either the wild type Fgl2 or a prothrombin cleaving-fragment thereof, polyclonal antisera or monoclonal antibodies can be made using standard methods. A mammal, (e.g., a mouse, hamster, or rabbit) can be immunized with an immunogenic form of the peptide which elicits an antibody response in the mammal. The amino acid sequence for human Fgl2, a useful immunogen for such purposes, is known from Levy et al, supra. Moreover, a technique for producing monoclonal anti-Fgl2 antibody is described by Levy et al, in J. Biol. Chem., 1991, 286(3):1789. The preparation of rabbit polyclonal antiserum against Fgl2 is described in Ding et al. (1997) J. Virol. 71(12):9223. Techniques for conferring immunogenicity on a peptide include conjugation to carriers or other techniques well known in the art. For example, the protein or peptide can be administered in the presence of adjuvant. The progress of immunization can be monitored by detection of antibody titers in plasma or serum. Standard ELISA or other immunoassay procedures can be used with the immunogen as antigen to assess the levels of antibodies. Following immunization, antisera can be obtained and, if desired, polyclonal antibodies isolated from the sera.

To produce monoclonal antibodies, antibody producing cells (lymphocytes) can be harvested from an immunized animal and fused with myeloma cells by standard somatic cell fusion procedures thus immortalizing these cells and yielding hybridoma cells. Such techniques are well known in the art, (e.g., the hybridoma technique originally developed by Kohler and Milstein (Nature 256, 495-497 (1975)) as well as other techniques such as the human B-cell hybridoma technique (Kozbor et al., Immunol. Today 4, 72 (1983)), the EBV-hybridoma technique to produce human monoclonal antibodies (Cole et al. Monoclonal Antibodies in Cancer Therapy (1985) Allen R. Bliss, Inc., pages 77-96), and screening of combinatorial antibody libraries (Huse et al., Science 246, 1275 (1989)). Hybridoma cells can be screened immunochemically for production of antibodies specifically reactive with the peptide and the monoclonal antibodies can be isolated. Therefore, the invention also contemplates hybridoma cells secreting monoclonal antibodies with specificity for Fgl2.

The term “antibody” as used herein is intended to include fragments thereof that also specifically bind with Fgl2 or a prothrombin-cleaving fragment or domain thereof. Antibodies can be fragmented using conventional techniques and the fragments screened for utility in the same manner as described above. For example, F(ab′)₂ fragments can be generated by treating antibody with pepsin. The resulting F(ab′)₂ fragment can be treated to reduce disulfide bridges to produce Fab′ fragments.

Chimeric antibody derivatives, i.e., antibody molecules that combine a non-human animal variable region and a human constant region are also contemplated within the scope of the invention. Chimeric antibody molecules can include, for example, the antigen binding domain from an antibody of a mouse, rat, or other species, with human constant regions. Conventional methods may be used to make chimeric antibodies containing the immunoglobulin variable region which recognizes the gene product of a CD200R antigens of the invention (See, for example, Morrison et al., Proc. Natl Acad. Sci. U.S.A. 81,6851 (1985); Takeda et al., Nature 314, 452 (1985), Cabilly et al., U.S. Pat. No. 4,816,567; Boss et al., U.S. Pat. No. 4,816,397; Tanaguchi et al., European Patent Publication EP171496; European Patent Publication 0173494, United Kingdom patent GB 2177096B). It is expected that chimeric antibodies would be less immunogenic in a human subject than the corresponding non-chimeric antibody.

Monoclonal or chimeric antibodies specifically reactive with a protein of the invention as described herein can be further humanized by producing human constant region chimeras, in which parts of the variable regions, particularly the conserved framework regions of the antigen-binding domain, are of human origin and only the hypervariable regions are of non-human origin. Such immunoglobulin molecules may be made by techniques known in the art, (e.g., Teng et al., Proc. Natl. Acad. Sci. U.S.A., 80, 7308-7312 (1983); Kozbor et al., Immunology Today, 4, 7279 (1983); Olsson et al., Meth. Enzymol., 92, 3-16 (1982)), and PCT Publication WO92/06193 or EP 0239400). Humanized antibodies can also be commercially produced (Scotgen Limited, 2 Holly Road, Twickenham, Middlesex, Great Britain.)

Specific antibodies, or antibody fragments, reactive against Fgl2 may also be generated by screening expression libraries encoding immunoglobulin genes, or portions thereof, expressed in bacteria with peptides produced from the nucleic acid molecules of Fgl2. For example, complete Fab fragments, VH regions and FV regions can be expressed in bacteria using phage expression libraries (See for example Ward et al., Nature 341, 544-546: (1989); Huse et al., Science 246, 1275-1281 (1989); and McCafferty et al. Nature 348, 552-554 (1990)). Alternatively, a SCID-hu mouse, for example the model developed by Genpharm, can be used to produce antibodies or fragments thereof.

Thus, as noted above, Fgl2 inhibitors useful in the present invention include antibodies and Fgl2-binding antibody fragments that inhibit, and desirably neutralize, Fgl2-mediated prothrombin cleavage. Such antibodies can be raised against wild type Fgl2, e.g., human Fgl2, or against fragments, domains or epitopes involved in mediating such cleavage. Fgl2 fragments useful as immunogen include those which incorporate the C-terminal portion of Fgl2. It has been found, for instance, that coagulation activity of Fgl2 is retained following truncation of about 100 C-terminal residues, i.e., is significantly reduced when only the first 284 residues of the 432-mer wild type protein are present, but is restored in fragments that incorporate the first 336 residues of Fgl2. Thus, desirably, the immunogen used to raise Fgl2 antibody desirably incorporates at least more than residues 1-284 of Fgl2, e.g., incorporate resides 1-336 and up to all of residues 1-432. Alternatively, the immunogen comprises at least about residues 300-432 of human Fgl2. It will also be appreciated that the immunogen can comprises cells per se that express a functional Fgl2 protein, such as transfected CHO cells, as well as cells that normally express Fgl2 such as macrophages.

It will further be appreciated that agents useful to inhibit the prothrombinase activity of Fgl2 are not limited to antibodies that bind Fgl2, but instead include agents that inactivate genetic expression of Fgl2 such as sense and anti-sense RNA and DNA constructs that bind genomic DNA or mRNA encoding Fgl2, as well as soluble Fgl2 fragments that compete with Fgl2 for binding to prothrombin, and the like.

Accordingly, the present invention includes the use of antisense oligonucleotides to inhibit the activity of Fgl2.

The term “antisense oligonucleotide” as used herein means a nucleotide sequence that is complementary to its target. The term “oligonucleotide” refers to an oligomer or polymer of nucleotide or nucleoside monomers consisting of naturally occurring bases, sugars, and intersugar (backbone) linkages. The term also includes modified or substituted oligomers comprising non-naturally occurring monomers or portions thereof, which function similarly. Such modified or substituted oligonucleotides may be preferred over naturally occurring forms because of properties such as enhanced cellular uptake, or increased stability in the presence of nucleases. The term also includes chimeric oligonucleotides which contain two or more chemically distinct regions. For example, chimeric oligonucleotides may contain at least one region of modified nucleotides that confer beneficial properties (e.g. increased nuclease resistance, increased uptake into cells), or two or more oligonucleotides of the invention may be joined to form a chimeric oligonucleotide.

The antisense oligonucleotides of the present invention may be ribonucleic or deoxyribonucleic acids and may contain naturally occurring bases including adenine, guanine, cytosine, thymidine and uracil. The oligonucleotides may also contain modified bases such as xanthine, hypoxanthine, 2-aminoadenine, 6-methyl, 2-propyl and other alkyl adenines, 5-halo uracil, 5-halo cytosine, 6-aza uracil, 6-aza cytosine and 6-aza thymine, pseudo uracil, 4-thiouracil, 8-halo adenine, 8-aminoadenine, 8-thiol adenine, 8-thiolalkyl adenines, 8-hydroxyl adenine and other 8-substituted adenines, 8-halo guanines, 8-amino guanine, 8-thiol guanine, 8-thiolalkyl guanines, 8-hydroxyl guanine and other 8-substituted guanines, other aza and deaza uracils, thymidines, cytosines, adenines, or guanines, 5-trifluoromethyl uracil and 5-trifluoro cytosine.

Other antisense oligonucleotides of the invention may contain modified phosphorous, oxygen heteroatoms in the phosphate backbone, short chain alkyl or cycloalkyl intersugar linkages or short chain heteroatomic or heterocyclic intersugar linkages. For example, the antisense oligonucleotides may contain phosphorothioates, phosphotriesters, methyl phosphonates, and phosphorodithioates. In an embodiment of the invention there are phosphorothioate bonds links between the four to six 3′-terminus bases. In another embodiment phosphorothioate bonds link all the nucleotides.

The antisense oligonucleotides of the invention may also comprise nucleotide analogs that may be better suited as therapeutic or experimental reagents. An example of an oligonucleotide analogue is a peptide nucleic acid (PNA) wherein the deoxyribose (or ribose) phosphate backbone in the DNA (or RNA), is replaced with a polyamide backbone which is similar to that found in peptides (P. E. Nielsen, et al Science 1991, 254, 1497). PNA analogues have been shown to be resistant to degradation by enzymes and to have extended lives in vivo and in vitro. PNAs also bind stronger to a complementary DNA sequence due to the lack of charge repulsion between the PNA strand and the DNA strand. Other oligonucleotides may contain nucleotides containing polymer backbones, cyclic backbones, or acyclic backbones. For example, the nucleotides may have morpholino backbone structures (U.S. Pat. No. 5,034,506). Oligonucleotides may also contain groups such as reporter groups, a group for improving the pharmacokinetic properties of an oligonucleotide, or a group for improving the pharmacodynamic properties of an antisense oligonucleotide. Antisense oligonucleotides may also have sugar mimetics.

The antisense nucleic acid molecules may be constructed using chemical synthesis and enzymatic ligation reactions using procedures known in the art. The antisense nucleic acid molecules of the invention or a fragment thereof, may be chemically synthesized using naturally occurring nucleotides or variously modified nucleotides designed to increase the biological stability of the molecules or to increase the physical stability of the duplex formed with mRNA or the native gene e.g. phosphorothioate derivatives and acridine substituted nucleotides. The antisense sequences may be produced biologically using an expression vector introduced into cells in the form of a recombinant plasmid, phagemid or attenuated virus in which antisense sequences are produced under the control of a high efficiency regulatory region, the activity of which may be determined by the cell type into which the vector is introduced.

The antisense oligonucleotides may be introduced into tissues or cells using techniques in the art including vectors (retroviral vectors, adenoviral vectors and DNA virus vectors) or physical techniques such as microinjection. The antisense oligonucleotides may be directly administered in vivo or may be used to transfect cells in vitro which are then administered in vivo.

### Reference to Experimental Work

The inventors chose to study chronic hepatitis B viral infection in the Asian-Pacific region, where chronic infection is especially prevalent and patients usually acquire the infection at the time of birth or early in childhood. Some of these patients, whose infection originated earlier in life, present in the 3^{rd} and 4^{th} decades of life with marked chronic hepatitis. This disease is characterized by recurrent flares of hepatocellular injury. Minimal chronic viral hepatitis B may reflect either clinical remission of the latter or more benign disease. Biopsy is necessary to distinguish the two disorders and has a direct influence on therapeutic choices. Flares of marked chronic hepatitis are postulated to reflect T-lymphocyte-mediated immune responses to the virus (27). Indeed, T cell-mediated immune responses also play a prominent role in other forms of hepatocellular injury—autoimmune hepatitis, hepatotoxins and concanavalin A hepatitis (28). It is not clear what explains the difference in whether patients present with marked chronic hepatitis versus minimal chronic viral hepatitis B or what mediates transition from one form to the other. Intrahepatic production of interferon-gamma figures prominently in how viral hepatitis B-specific T cells mediate hepatocellular injury in marked chronic hepatitis (1, 29, 30). The current study demonstrates that expression of Fgl2/fibroleukin mRNA and protein varies markedly in patients with marked chronic hepatitis B versus those with minimal chronic viral hepatitis B. Expression of Fgl2/fibroleukin is highly correlated with fibrin expression. Taken together with results reported here for marked chronic hepatitis, pharmacologic blockade of Fgl2/fibroleukin offer an important newer treatment approach in hepatitis viral B-induced disease. It is important to stress that these findings identify that an association exists between expression of Fgl2/fibroleukin, a prothrombinase, and the fibrin deposition that attends disease in marked chronic hepatitis B.

To identify a cause and effect relationship between the Fgl2/fibroleukin prothrombinase and hepatocellular injury in viral hepatitis the inventors developed Fgl2/fibroleukin^{+/-} and Fgl2/fibroleukin^{-/-} mice and examined the effect of Fgl2/fibroleukin genotype on in vitro and in vivo responses to murine hepatitis virus type 3 (MHV-3). Macrophages from Fgl2/fibroleukin^{+/+} mice responded to the MHV-3 virus with a robust procoagulant response. In contrast, macrophages from Fgl2/fibroleukin^{-/-} mice generated no increase in procoagulant above control levels. LPS induced a similar procoagulant response in both. LPS is known to induce TF/Factor VII-dependent procoagulant activity in peritoneal macrophages. In the presence of calcium TF/Factor VIIa activates the conversion of the zymogen precursor Factor X into Factor Xa, the classical activator of thrombin generation. Using purified recombinant Fgl2/fibroleukin prothrombinase generated in a baculovirus system and reconstituted into phospholipids-containing vesicles, and MHV-3-infected murine peritoneal macrophages, the inventors recently assessed rates of thrombin generation using one-stage clotting assays, ¹²⁵I-labeled prothrombin cleavage assays and a chromogenic substrate of thrombin, namely H-D-hexahydrotyrosyl-L-alanyl-L-arginine-p-nitroanalide-diacetate. Like Factor Xa, the Fgl2/fibroleukin prothrombinase is dependent on calcium, phospholipids, and Factor Va for full activity (10). Fgl2/fibroleukin prothrombinase differs from Factor Xa in that activity on cell surfaces of MHV-3 infected peritoneal macrophages is resistant to extensive washing, consistent with the predicted transmembrane structure of the Fgl2/fibroleukin prothrombinase. Activity is not inhibited by antithrombin III and is not dependent on Factor VII. The Fgl2/fibroleukin prothrombinase generates a unique thrombin cleavage fragment of ≈24 kDa (10). Neutralizing antibodies directed against Fgl2/fibroleukin inhibit MHV-3-induced changes in the one-stage clotting assay (31). Such antibodies are specific in that they do not react with purified MHV-3 or LPS-induced procoagulants.

Mortality in MHV-3 infected Fgl2/fibroleukin^{-/-} mice was significantly reduced compared to Fgl2/fibroleukin^{+/+} littermates. Therefore infection of Fgl2/fibroleukin^{-/-} and Fgl2/fibroleukin^{+/+} mice tended to recapitulate the resistant and susceptible phenotypes previously reported for C57Bl/6 and A/J inbred strains, respectively (16). Also, targeted ablation of the Fgl2/fibroleukin gene and neutralizing antibodies against the Fgl2/fibroleukin prothrombinase resulted in comparable effects on MHV-3 pathogenesis (23). The response to LPS was not modified by Fgl2/fibroleukin genotype and contrasted with the effect in the LPS-hyporesponsive C3H/HeJ strain. When the inventors assessed fibrin deposition in the liver and quantified hepatocellular necrosis the inventors observed effects of MHV-3 infection that were consistent with the effect of the Fgl2/fibroleukin genotype on overall mortality, namely markedly decreased fibrin deposition and hepatocellular injury in Fgl2/fibroleukin^{-/-} mice. Previous studies have suggested the existence of multiple genetic modifiers for the MHV-3 induced fulminant hepatitis phenotype (19), including the CEACAM1 MHV viral receptor glycoprotein (32). MHV-3 viral loads in the livers did not vary with the Fgl2/fibroleukin genotype. This contrasts with the effects of targeted disruption of the CEACAM1 MHV viral receptor which modifies hepatotrophic host/viral interactions and decreases viral loads in the livers of MHV-3 infected mice (33). The inventors did not detect an important effect of ablating the Fgl2/fibroleukin gene on MHV-3 viral loads in the liver. To the extent that MHV-3 viral hepatitis can be used to model viral hepatitis B in humans, these cumulative findings can be taken to indicate that hepatocellular injury induced by MHV-3 is critically dependent on the Fgl2/fibroleukin prothrombinase.

The results provide compelling evidence for a role of the Fgl2/fibroleukin prothrombinase in human viral hepatitis B.

The following non-limiting examples are illustrative of the present invention:

### EXAMPLES

### Example 1

All animal experiments in the study were carried out according to the guidelines of the Canadian Council on Animal Care and approved by the University of Toronto and/or University Health Network Committees on Animal Experimentation. Informed consent was obtained from human study participants, and the research protocol was reviewed and approved by the institutional review board of Tonji Hospital, Wuhan, PRC.

Generation of Fgl2/fibroleukin prothrombinase deficient mice. The targeting construct was derived from a mouse 129Sv/J strain genomic library and transfected into 129Sv/J embryonic stem (ES) cells by electroporation. Appropriately targeted ES clones were injected into C57Bl/6 blastocysts. Chimeric males were mated with C57Bl/6 females. Mice were interbred from successive generations of Fgl2/fibroleukin^{+/-} founders and thus littermates represent a mixed 129Sv/J×C57Bl/6 genetic background. Southern blot analysis and multiplex PCR was used to genotype animals (details of probes and oligonucleotides are available from the authors upon request). The 129Sv/J inbred strain and the C57Bl/6 inbred strain are both MHV-3 sensitive (16). To assess the effects of strain background on loss of Fgl2/fibroleukin^{-/-} homozygotes and response to MHV-3 infection, Fgl2/fibroleukin^{+/-} mice were backcrossed with C57Bl/6 inbred mice for ten successive generations. The fidelity of the backcross was assessed by genotyping Fgl2/fibroleukin^{+/-} mice derived from the 10^{th} generation for two informative DNA markers per chromosome (i.e. different alleles in 129Sv/J versus C57Bl/6). Unless stated otherwise all studies were performed on the mixed 129Sv/J×C57Bl/6 genetic background.

Embryo studies. Uteri were removed from euthanized pregnant mice from 7.5 to 11.5 days pc and were fixed in freshly prepared buffered 4% paraformaldehyde for 18 hr then embedded in paraffin. Serial sections were cut and fixed to Aptex-coated slides. Every 10^{th} slide was stained with haematoxylin and eosin. Adjacent sections were stained with polyclonal rabbit anti-Fgl2 at 1/10,000 dilution or control rabbit IgG 1.25 μg/ml as described (17) without microwave treatment.

Hematological studies in mice. Mice were anesthetized followed by percutaneous cardiac puncture for blood aspiration. Approximately 500 μL of blood was collected into an EDTA-containing Microtainer tube (Becton-Dickinson, Franklin Lakes, N.J.). Hemoglobin level, platelet count, leukocyte count were performed by the University Health Network Medical Laboratories (Toronto, Ontario), where the automated equipment were re-adjusted to standardize for mouse samples. The Hemochron Jr. II Microcoagulation System (International Technidyne Corp., Edison, N.J.) was used for immediate measurement of prothrombin time (PT) and activated partial thromboplastin times (aPTT). Bleeding times were measured as described (18).

Procoagulant activity and Fgl2 expression in mice. Thioglycollate-elicited macrophages (4×10⁶/mL ) were exposed in vitro to MHV-3 (MOI 1), 10 μg/mL lipopolysaccharide (E coli serotype 0111, Sigma) or vehicle for 8 hr for procoagulant activity measurements. After three cycles of freeze-thawing and sonication, extracts were assayed for the capacity to shorten the spontaneous clotting time of normal citrated human plasma in a one stage clotting assay, as previously described (10). The time for appearance of a fibrin gel was recorded. First strand cDNA was synthesized using the SuperScript Preamplification protocol (Gibco BRL/Life Technologies, Rockville, Md.) followed by RT-PCR amplification using a set of Fgl2 specific primers spanning exon 1 and 2 (sense: 5′-TGC TCA AAG AAG TGC GGA CCC T-3′, antisense: 5′-GCC CCA CTG CTT CTC CTT CCT-3′, 430 bp). RT-PCR was also performed in the in vivo setting using tissues harvested from mice injected intraperitoneally with 1000 plaque-forming units (PFU) of MHV-3. Total cellular RNA was isolated 16 hr post infection and RT-PCR was performed as described above.

Susceptibility to MHV-3 infection and lipopolysaccharide exposure. For lipopolysaccharide (LPS) studies mice (n=10 per group, age 3 to 6 months) received LPS (i.p., 30 mg/kg E coli serotype O55:B5) as well as buprenorphine and saline for analgesia and fluid resuscitation (s.c. 0.1 mg/kg in 2 ml saline at t=0 and 12 hr), respectively. Mice were monitored every 2 hr for 24 hr for morbidity and mortality. Any animals judged to be suffering were euthanized by cervical dislocation. C57Bl/6 and C3H HeJ strains were used as LPS-sensitive and LPS-hyporesponsive mouse controls, respectively. For MHV-3 studies, mice (n=10 per group, age 3 to 6 months) were infected via i.p. injection with 1000 PFU of MHV-3. Multiple independent experiments were performed using different preparations of viral stocks. Following infection, mice were housed in pre-sterilized cages in an isolated environment and given food and water ad libitum. Mortality was scored when animals become moribund. Livers were removed from all euthanized mice and analysed for viral titers, liver histology and fibrin deposition. Frozen liver tissue was homogenized and viral titers determined on monolayers of L2 cells in a standard plaque assay (19). For histological examination and quantification of necrosis and fibrin deposition, liver tissue was fixed in 10% buffered formalin for 4 hr followed by routine tissue processing and staining using an automated tissue processor following which the tissue blocks were embedded in paraffin. Livers were then sectioned at 5 μm thickness and stained with haematoxylin and eosin and with MSB. Liver sections were immunostained for fibrin using the Nexes immunostaining method, an automated avidin-biotin method utilizing protease 1 treatment (Ventana Medical Systems, Tuscon, Ariz.) in which a purified antibody (a specific biotinylated Vector Labs 1:100 diluted anti rabbit IgG) was used. Sections were scored in a blinded fashion to determine the extent of necrosis and fibrin deposition. The surface area of the liver examined was equal in all instances and corresponded to 10 contiguous histological fields at low magnification (×100). Sera from all animals were analysed quantitatively for alanine aminotransferase (ALT) using a Worthington Statzyme GTP kit obtained from Cooper Biochemical as previously described (19).

Studies in patients with viral hepatitis B. Patients were recruited at Tonji Hospital, Wuhan, PRC, and are described in Table 2. Biochemical, histologic and clinical features were used to define patients with chronic viral hepatitis B: marked chronic viral hepatitis B versus minimal chronic viral hepatitis B. Diagnostic criteria and laboratory investigations used standard definitions (20). The evaluation of patients with chronic viral hepatitis B infection included a thorough history and physical examination, with special emphasis on risk factors for co-infection (with hepatitis C, D or HIV), alcohol use, and family history of viral hepatitis B infection and liver cancer. Percutaneous liver biopsies were obtained for the purposes of guiding treatment decisions (21). Hepatitis B viral markers and serology (HbsAg, HbeAg, anti-HbcAg) were assessed using ELISA assay kits obtained from Shanghai Shi yue Ke Hua Biological Science Inc., Shanghai, PRC and/or Abbott Laboratories, Illinois, according to the manufacturer's instructions. Hepatitis B viral DNA loads were assessed by PCR (sense: 5′-TTT GGG GCA TGG ACA TTG AC-3′, antisense: 5′-CCC ACC TTA TGA GTC CAA GG-3′). Twenty-three patients with marked chronic hepatitis B and thirteen cases of minimal chronic viral hepatitis B were evaluated. Sections of liver tissue were scored in a blinded fashion as to the histological diagnosis, fibrin deposition, and Fgl2/fibroleukin immunostaining. For fibrin detection, a rabbit-anti-human fibrinogen antibody (DAKO, Carpenteria, Calif.) was used which is known to react with fibrinogen and fibrin in human tissues (7). A CD68 antibody (DAKO) was used to detect macrophages (Kupffer cells). For Fgl2/fibroleukin immunostaining, the R4 polyclonal rabbit antibody was used as described (17). Immunoperoxidase staining (dark brown) used the avidin-biotin complex method. For the in situ hybridization studies a 169-bp DNA fragment from the carboxyl end of the human Fgl2/fibroleukin coding region was used as a template to synthesize anti-sense digoxigenin-11-UTP-labeled cRNA probes (Hoffman-La Roche, Basel, Switzerland) and detected using the alkaline phosphatase method, as described (7).

Statistical Analyses. All data are reported as the mean value±SEM, with sample sizes (n) stated throughout. Mendelian heritability and survival curves were statistically assessed using the Chi-squared test with Fisher's-exact test, and Kaplan-Meier survival analysis, respectively (StatView V4.5; Abacus Concepts Inc., San Francisco, Calif., USA). P-values<0.05 were considered significant.

### RESULTS

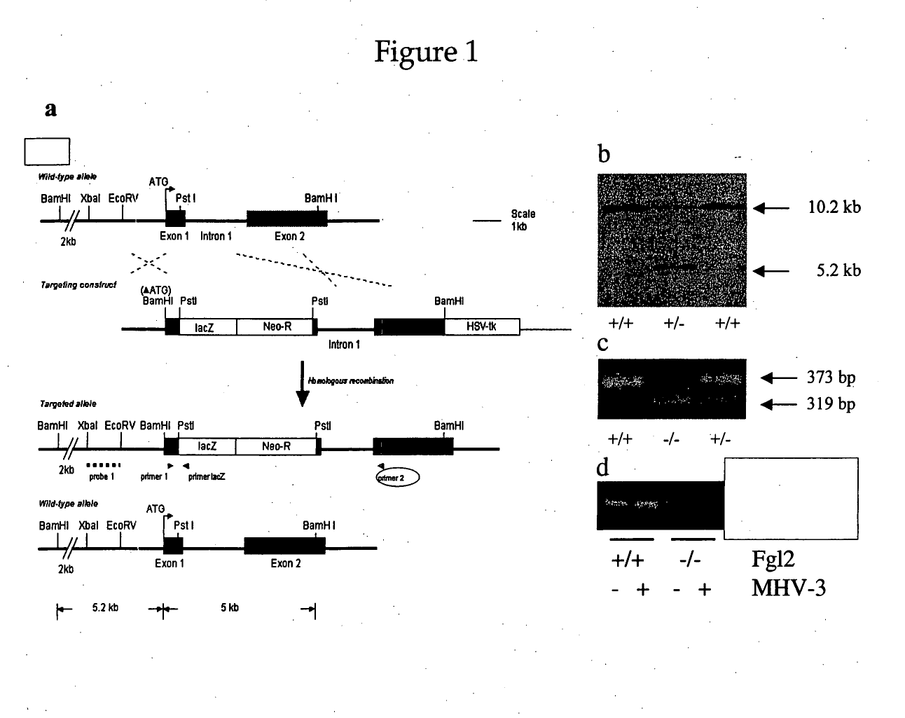

Generation of Fgl2/fibroleukin prothrombinase deficient mice. To investigate the in vivo role(s) for Fgl2/fibroleukin in the procoagulant response in disease the inventors created and analyzed Fgl2/fibroleukin-deficient mice. A targeting vector for functional Fgl2 gene ablation (FIG. 1a) was constructed by insertion of a LacZ reporter and PGK-neo gene within the first of two coding exons and mutagenesis of the Fgl2/fibroleukin initiator methionine codon in exon 1. Chimeric mice were generated by injection of targeted embryonic stem (ES) cell clones (FIG. 1b) into C57Bl/6-derived blastocysts by standard approaches. Fgl2/fibroleukin^{+/-} intercrosses produced viable Fgl2/fibroleukin^{-/-} offspring as identified by Southern blotting and multiplex PCR (FIG. 1c).

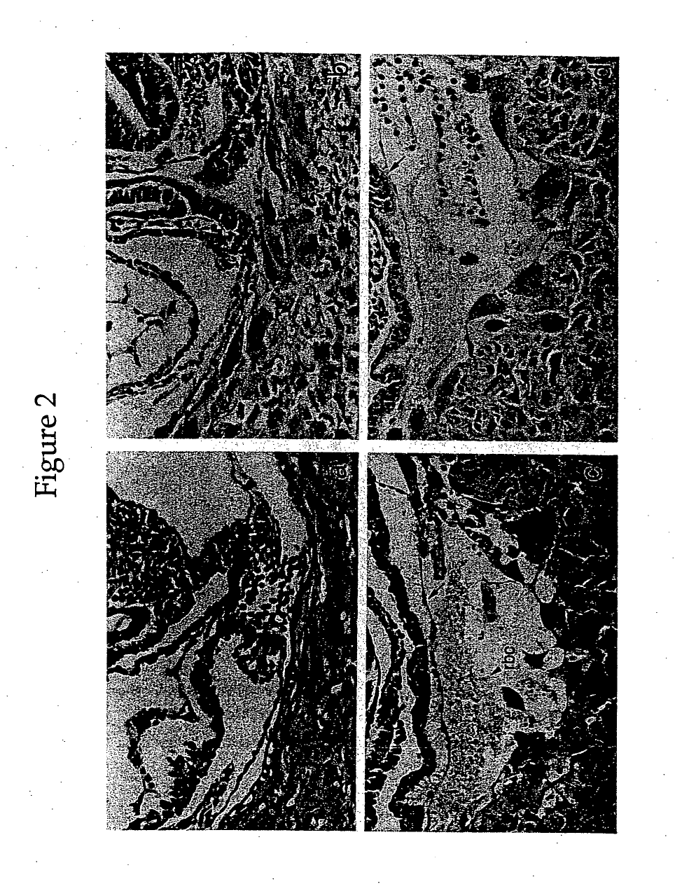

Hemorrhage at the fetal/maternal interface in Fgl2/fibroleukin^{+/-} mice. Interbreedings of Fgl2/fibroleukin^{+/-} mice produced offspring at ratios that deviated from the expected Mendelian ratios. At weaning, genotyping identified 156, 269, and 86 Fgl2/fibroleukin +/+, +/− and −/− mice, respectively (observed ratio 1:1.7:0.6 versus expected 1:2:1 ratio, _{χ}2 20.6, p<0.001). Importantly, 40% of Fgl2/fibroleukin^{-/-} homozygotes were lost. At 10.5 days post coitum (pc), genotyping of embryos indicated that an important degree of embryo loss was occurring early in development. To determine when embryo losses occurred, zygotes and uterine tissue were examined by light microscopy and immunohistochemistry using a polyclonal anti-Fgl2 antibody from 7.5 to 11.5 days pc. On day 7.5 pc embryos and uterine decidua appeared normal. Starting at day 8.5 pc many of the Fgl2/fibroleukin^{-/-} embryos showed moderate to severe hemorrhage at implantation sites with extravasation of maternal erythrocytes between the mural trophoectoderm and Reichert's membrane, especially at the anti-mesometrial pole (FIG. 2). Immunohistochemistry showed embryos with hemorrhages were Fgl2/fibroleukin-deficient in mural trophectoderm in contrast to embryos without hemorrhages and to maternal decidua (FIG. 2). Embryonic or maternal hemorrhage was otherwise not observed early or late in pregnancy. The loss of Fgl2/fibroleukin ^{-/-} homozygotes was not modified significantly on the C57Bl/6 inbred background. When Fgl2/fibroleukin^{+/-} mice were backcrossed with C57Bl/6 inbred mice through ten successive generations and intercrosses of Fgl2/fibroleukin^{+/-} repeated, the inventors again detected loss of Fgl2/fibroleukin ^{-/-} homozygotes. At weaning the inventors detected 62 and 14 Fgl2/fibroleukin +/− and −/− mice, respectively (observed ratio 1:0.2 versus expected 1:0.5 ratio, _{χ}2 7.6, p<0.01).

Standard hematological profiles (hemoglobin, white blood cell count and platelet counts), bleeding times, and prothrombin and partial thromboplastin time estimates in Fgl2/fibroleukin-deficient adult mice were similar to littermate controls (data not shown). Adult Fgl2/fibroleukin^{-/-} mice were unremarkable compared to littermates with respect to gross anatomical examination, bleeding episodes or longevity (2.5 years observation).

Procoagulant activity (PCA) and Fgl2/fibroleukin expression. Murine peritoneal macrophages infected in vitro with MHV-3 express a unique cell-surface calcium- and phospholipid-dependent procoagulant with direct prothrombinase activity. Cloning and heterologous expression of the MHV-3-induced procoagulant identified Fgl2/fibroleukin (6, 8, 10). Strain-dependent susceptibility to MHV-3-induced fulminant hepatic failure correlates with the ability of TH₁-type lymphocytes to induce expression of Fgl2/fibroleukin (22). Whereas the A/J strain of mice is resistant to fulminant hepatic failure, the C57Bl/6 and CV129 strains are fully susceptible to MHV-3 and die within 4-7 days (23). Importantly, MHV-3 infection failed to induce the Fgl2/fibroleukin mRNA (FIG. 1d) or procoagulant activity (Table 1) in thioglycollate-elicited peritoneal macrophages isolated from Fgl2/fibroleukin^{-/-} mice. LPS is known to induce TF/Factor VII-dependent procoagulant activity in peritoneal macrophages. In contrast to the abrogation of MHV-3-induced procoagulant activity, the response to LPS was unaffected in Fgl2/fibroleukin^{-/-} cells (Table 1).

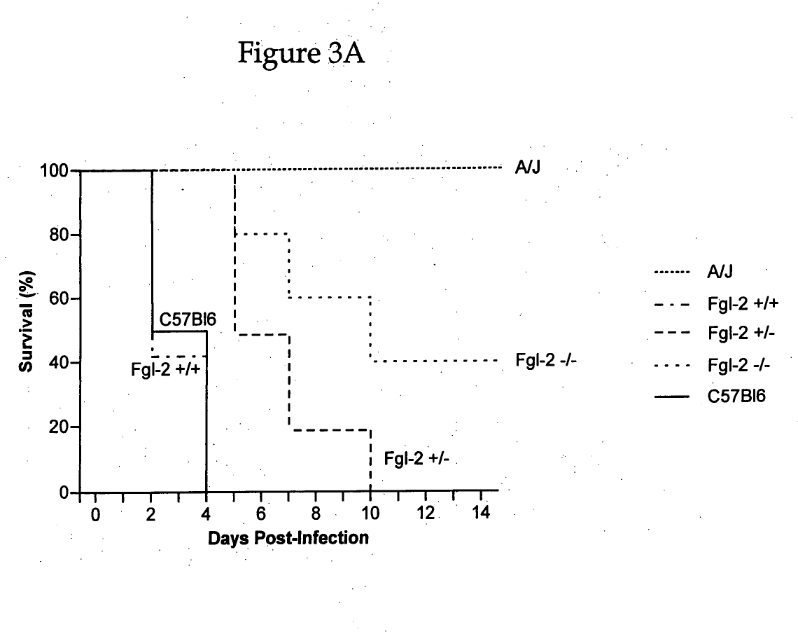

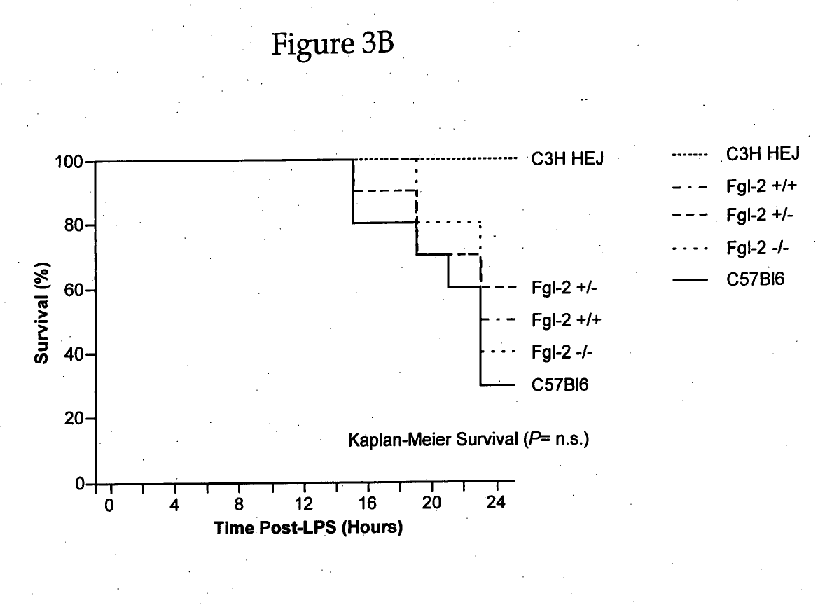

Susceptibility to MHV-3 infection and lipopolysaccharide exposure in Fgl2/fibroleukin^{-/-} mice. Mortality in MHV-3 infected mice (1000 pfu) Fgl2/fibroleukin^{-/-} mice was significantly blunted compared to Fgl2/fibroleukin^{+/+} littermates (FIG. 3a), with heterozygotes showing an intermediate phenotype. Infection of A/J and C57Bl/6 mice recapitulated the resistant and susceptible phenotypes previously reported (16). Resistance of Fgl2/fibroleukin^{-/-} mice to MHV-3-induced death was reproducible in multiple independent infections and across different preparations of viral stocks. The inventors assessed the effects of strain background on MHV-3 induced liver necrosis and survival in mice infected with MHV-3. Consistent with findings in the mixed 129Sv/J×C57Bl/6 genetic background, findings indicated that Fgl2/fibroleukin (−/−) mice on the C57Bl/6 background were also resistant to MHV-3 induced liver necrosis and death (data not shown). The response of Fgl2/fibroleukin^{-/-} mice to systemic inflammation induced by acute exposure to LPS was defined and compared to the LPS-hyporesponsive C3H/HeJ strain. All mouse strains, except for the C3H/HeJ, exhibited signs of illness (mottled fur, inactivity, lethargy). The Fgl2/fibroleukin genotype failed to modify this response. Also there was no difference in survival between non-C3H/HeJ strains by Kaplan Meier analysis demonstrating that disruption of the Fgl2 gene does not affect systemic LPS responses (FIG. 3b).

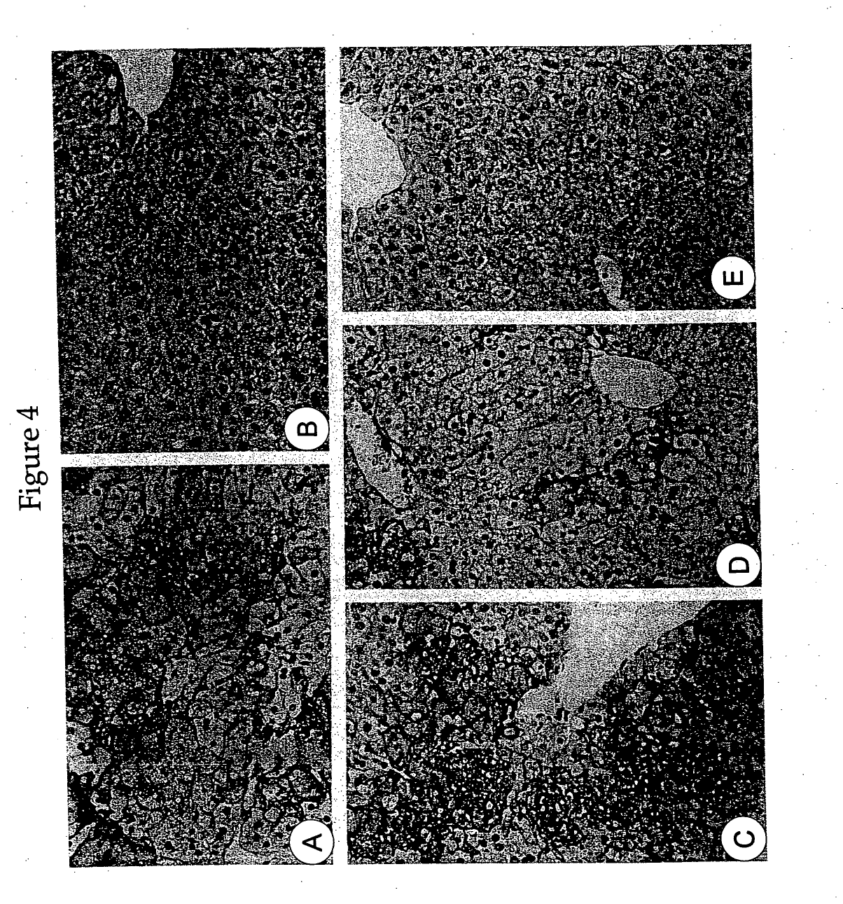

When the inventors assessed fibrin deposition in the liver and quantified hepatocellular necrosis in the viral hepatitis model (FIG. 4) the inventors observed effects of MHV-3 infection that were consistent with the phenotypic effect of the Fgl2/fibroleukin genotype on overall mortality, namely markedly decreased fibrin deposition and liver cell necrosis in Fgl2/fibroleukin^{-/-} mice. Marked elevations in serum alanine aminotransferase (ALT) levels, a biochemical marker of liver cell histopathology (baseline values in normal mice<50 IU/L), were seen three days after viral infection in C57/Bl6 and Fgl2/fibroleukin^{+/+} mice (7420 IU/L, 6490 IU/L). This can be contrasted with the trivial injury observed in A/J and Fgl2/fibroleukin^{-/-} mice (54 IU/L, 87 IU/L) or the intermediate response observed in Fgl2/fibroleukin^{+/-} mice (870 IU/L). When assessed at 14 days, modest elevations in ALT levels were evident in surviving Fgl2/fibroleukin^{-/-} mice compared to the A/J resistant strain (625 versus 80 IU/L, p<0.05). MHV-3 viral loads in the livers of A/J, Fgl2/fibroleukin^{-/-}, Fgl2/fibroleukin^{+/+} and C57/Bl6 mice three days after infection were 8.0, 9.0, 8.2 and 9.0×10⁷ pfu/gm tissue indicating that the Fgl2/fibroleukin genotype did not have a major effect on the production of infectious virus in the liver. Fourteen days after infection, MHV-3 viral loads were significantly higher in surviving Fgl2/fibroleukin^{-/-} mice compared to the resistant A/J strain (6.0×10⁹ versus 2.0×10² pfu/gm liver tissue). This indicates that although Fgl2/fibroleukin^{-/-} mice evidenced an important degree of protection against MHV-3-induced fulminant hepatic failure that, in contrast to the resistant A/J strain, they failed to eliminate the virus.

Increased Fgl2/fibroleukin mRNA and protein in chronic active hepatitis B. To address the relevance of Fgl2/fibroleukin in human viral hepatitis, the inventors studied patients with chronic hepatitis B. The inventors have previously reported the co-expression of Fgl2/fibroleukin mRNA transcripts and fibrin in the livers of three patients with acute fulminant hepatic failure due to hepatitis B virus, an uncommon but devastating illness (7). In contrast, chronic hepatitis B virus infection is a common disease with worldwide distribution. This serious clinical problem has protean adverse sequelae, including hepatic cirrhosis and hepatocellular carcinoma. Hepatitis B virus infection is especially endemic in Asia. Twenty-three patients with biochemical, histologic and clinical evidence of marked chronic viral hepatitis B and thirteen patients with minimal chronic viral hepatitis B were studied for hepatic Fgl2/fibroleukin expression. Patient characteristics are noted in Table 2. Of note HbsAg, HbeAg, anti-HBcAg and hepatitis B viral DNA load did not correlate with expression of Fgl2/fibroleukin or fibrin deposition. Though alanine aminotransferase (ALT) and total bilirubin (TBil) levels tended to be higher in patients with marked chronic hepatitis versus minimal chronic hepatitis B, there was considerable overlap in patient values. Twenty-one of twenty-three patients with marked chronic viral hepatitis B were positive for Fgl2/fibroleukin expression, both by in situ hybridization and immunohistochemistry. Fgl2/fibroleukin transcripts and immunoreactive protein were seen in macrophages and endothelial cells in areas of hepatic inflammation and necrosis both in portal areas and in the sinusoids (FIG. 5). Associated with the Fgl2/fibroleukin expression was fibrin deposition, as shown in FIG. 5. Of the thirteen patients with minimal chronic viral hepatitis B (Table 2), one patient demonstrated Fgl2/fibroleukin and fibrin expression. CD68 positive cells (Kupffer/macrophages) were clearly evident in all patients.

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

TABLE 1
Plasma clotting time (in seconds) by macrophages harvested
from Fgl2 +/+, +/−, or −/− mice stimulated with MHV-3 or LPS

| | Fgl2/fibroleukin Genotype | | |
|---|---|---|---|
| Stimulus | +/+ | +/− | −/− |
| None | ≧120 | ≧120 | ≧120 |
| MHV-3 (1000 pfu) | 34 ± 3 | 41 ± 2 | ≧120 |
| LPS (10 μg) | 36 ± 7 | 38 ± 5 | 35 ± 4 |

TABLE 2
Characteristics of the Patients

| | Minimal | Marked |
|---|---|---|
| | Chronic | Chronic |
| | Hepatitis B | Hepatitis B |
| Characteristic | (n = 13) | (n = 23) |
| Age (yr) | | |
| Mean | 43.8 | 36.0 |
| S.D. | 5.6 | 7.8 |
| Male sex (%) | 85 | 87 |
| HBsAg (%) | 100 | 100 |
| HBeAg (%) | 46 | 30 |
| Anti HBcAg (%) | 85 | 91 |
| HBV DNA* | 54 | 39 |
| Serum alanine aminotransferase (I.U./litre) | | |
| Mean | 300.5 | 586.0 |
| S.D. | 238.9 | 404.2 |
| Serum bilirubin (micromoles/litre)§ | | |
| Mean | 78.3 | 452.1 |
| S.D. | 89.4 | 191.7 |

*The limit of detection of this assay was approximately 10 genomes
§To convert values for bilirubin to mg/dl, divide by 17.1

### REFERENCES

1. Guidotti, L. G., and Chisari, F. V. 1996. To kill or to cure: options in host defense against viral infection. Curr Opin Immunol 8:478-483.
2. Levy, G. A., MacPhee, P. J., Fung, L. S., Fisher, M. M., and Rappaport, A. M. 1983. The effect of mouse hepatitis virus infection on the microcirculation of the liver. Hepatology 3:964-973.
3. Mackman, N. 1997. Regulation of the tissue factor gene. Thromb Haemost 78:747-754.
4. Osterud, B., and Bjorklid, E. 2001. The tissue factor pathway in disseminated intravascular coagulation. Semin Thromb Hemost 27:605-617.
5. Levy, G. A., Leibowitz, J. L., and Edgington, T. S. 1981. Induction of monocyte procoagulant activity by murine hepatitis virus type 3 parallels disease susceptibility in mice. J Exp Med 154:1150-1163.
6. Parr, R. L., Fung, L., Reneker, J., Myers-Mason, N., Leibowitz, J. L., and Levy, G. 1995. Association of mouse fibrinogen-like protein with murine hepatitis virus-induced prothrombinase activity. J Virol 69:5033-5038.
7. Levy, G. A., Liu, M., Ding, J., Yuwaraj, S., Leibowitz, J., Marsden, P. A., Ning, Q., Kovalinka, A., and Phillips, M. J. 2000. Molecular and functional analysis of the human prothrombinase gene (HFGL2) and its role in viral hepatitis. Am J Pathol 156:1217-1225.
8. Yuwaraj, S., Ding, J., Liu, M., Marsden, P. A., and Levy, G. A. 2001. Genomic characterization, localization, and functional expression of FGL2, the human gene encoding fibroleukin: a novel human procoagulant. Genomics 71:330-338.
9. Ruegg, C., and Pytela, R. 1995. Sequence of a human transcript expressed in T-lymphocytes and encoding a fibrinogen-like protein. Gene 160:257-262.
10. Chan, C. W., Chan, M. W., Liu, M., Fung, L., Cole, E. H., Leibowitz, J. L., Marsden, P. A., Clark, D. A., and Levy, G. A. 2002. Kinetic analysis of a unique direct prothrombinase, fgl2, and identification of a serine residue critical for the prothrombinase activity. J Immunol 168:5170-5177.
11. Koyama, T., Hall, L. R., Haser, W. G., Tonegawa, S., and Saito, H. 1987. Structure of a cytotoxic T-lymphocyte-specific gene shows a strong homology to fibrinogen beta and gamma chains. Proc Natl Acad Sci USA 84:1609-1613.
12. Qureshi, S. T., Clermont, S., Leibowitz, J., Fung, L. S., Levy, G., and Malo, D. 1995. Mouse hepatitis virus-3 induced prothrombinase (Fgl2) maps to proximal chromosome 5. Genomics 29:307-309.
13. Lafuse, W. P., Castle, L., Brown, D., and Zwilling, B. S. 1995. The cytotoxic T lymphocyte gene FIBLP with homology to fibrinogen beta and gamma subunits is also induced in mouse macrophages by IFN-gamma. Cell Immunol 163:187-190.
14. Marazzi, S., Blum, S., Hartmann, R., Gundersen, D., Schreyer, M., Argraves, S., von Fliedner, V., Pytela, R., and Ruegg, C. 1998. Characterization of human fibroleukin, a fibrinogen-like protein secreted by T lymphocytes. J Immunol 161:138-147.
15. Ding, J. W., Ning, Q., Liu, M. F., Lai, A., Leibowitz, J., Peltekian, K. M., Cole, E. H., Fung, L. S., Holloway, C., Marsden, P. A., et al. 1997. Fulminant hepatic failure in murine hepatitis virus strain 3 infection: tissue-specific expression of a novel fgl2 prothrombinase. J Virol 71:9223-9230.
16. MacPhee, P. J., Dindzans, V. J., Fung, L. S., and Levy, G. A. 1985. Acute and chronic changes in the microcirculation of the liver in inbred strains of mice following infection with mouse hepatitis virus type 3. Hepatology 5:649-660.
17. Knackstedt, M., Ding, J. W., Arck, P. C., Hertwig, K., Coulam, C. B., August, C., Lea, R., Dudenhausen, J. W., Gorczynski, R. M., Levy, G. A., et al. 2001. Activation of the novel prothrombinase, fgl2, as a basis for the pregnancy complications spontaneous abortion and pre-eclampsia. Am J Reprod Immunol 46:196-210.
18. Law, D. A., DeGuzman, F. R., Heiser, P., Ministri-Madrid, K., Killeen, N., and Phillips, D. R. 1999. Integrin cytoplasmic tyrosine motif is required for outside-in alphaIIbbeta3 signalling and platelet function. Nature 401:808-811.
19. Dindzans, V. J., Skamene, E., and Levy, G. A. 1986. Susceptibility/resistance to mouse hepatitis virus strain 3 and macrophage procoagulant activity are genetically linked and controlled by two non-H-2-linked genes. J Immunol 137:2355-2360.
20. Phillips, M. J., Cameron, R., Flowers, M. A., Blendis, L. M., Greig, P. D., Wanless, I., Sherman, M., Superina, R., Langer, B., and Levy, G. A. 1992. Post-transplant recurrent hepatitis B viral liver disease. Viral-burden, steatoviral, and fibroviral hepatitis B. Am J Pathol 140:1295-1308.
21. Lok, A. S., Heathcote, E. J., and Hoofnagle, J. H. 2001. Management of hepatitis B: 2000—summary of a workshop. Gastroenterology 120:1828-1853.
22. Liu, M. F., Ning, Q., Pope, M., Mosmann, T., Leibowitz, J., Ding, J. W., Fung, L. S., Rotstein, O., Gorczynski, R., and Levy, G. A. 1998. Resistance of naive mice to murine hepatitis virus strain 3 requires development of a Th1, but not a Th2, response, whereas pre-existing antibody partially protects against primary infection. Adv Exp Med Biol 440:415-423.
23. Li, C., Fung, L. S., Chung, S., Crow, A., Myers-Mason, N., Phillips, M. J., Leibowitz, J. L., Cole, E., Ottaway, C. A., and Levy, G. 1992. Monoclonal antiprothrombinase (3D4.3) prevents mortality from murine hepatitis virus (MHV-3) infection. J Exp Med 176:689-697.
24. Maupas, P., and Melnick, J. L. 1981. Hepatitis B infection and primary liver cancer. Prog Med Virol 27:1-5.
25. Hoofnagle, J. H., and di Bisceglie, A. M. 1997. The treatment of chronic viral hepatitis. N Engl J Med 336:347-356.
26. Pawlotsky, J. M. 2002. Molecular diagnosis of viral hepatitis. Gastroenterology 122:1554-1568.
27. Guidotti, L. G., and Chisari, F. V. 2001. Noncytolytic control of viral infections by the innate and adaptive immune response. Annu Rev Immunol 19:65-91.
28. Hong, F., Jaruga, B., Kim, W. H., Radaeva, S., El-Assal, O. N., Tian, Z., Nguyen, V. A., and Gao, B. 2002. Opposing roles of STAT1 and STAT3 in T cell-mediated hepatitis: regulation by SOCS. J Clin Invest 110:1503-1513.
29. Leifeld, L., Cheng, S., Ramakers, J., Dumoulin, F. L., Trautwein, C., Sauerbruch, T., and Spengler, U. 2002. Imbalanced intrahepatic expression of interleukin 12, interferon gamma, and interleukin 10 in fulminant hepatitis B. Hepatology 36:1001-1008.
30. Kakimi, K., Lane, T. E., Wieland, S., Asensio, V. C., Campbell, I. L., Chisari, F. V., and Guidotti, L. G. 2001. Blocking chemokine responsive to gamma-2/interferon (IFN)-gamma inducible protein and monokine induced by IFN-gamma activity in vivo reduces the pathogenetic but not the antiviral potential of hepatitis B virus-specific cytotoxic T lymphocytes. J Exp Med 194:1755-1766.
31. Fung, L. S., Neil, G., Leibowitz, J., Cole, E. H., Chung, S., Crow, A., and Levy, G. A. 1991. Monoclonal antibody analysis of a unique macrophage procoagulant activity induced by murine hepatitis virus strain 3 infection. J Biol Chem 266:1789-1795.
32. Ohtsuka, N., and Taguchi, F. 1997. Mouse susceptibility to mouse hepatitis virus infection is linked to viral receptor genotype. J Virol 71:8860-8863.
33. Blau, D. M., Turbide, C., Tremblay, M., Olson, M., Letourneau, S., Michaliszyn, E., Jothy, S., Holmes, K. V., and Beauchemin, N. 2001. Targeted disruption of the Ceacam1 (MHVR) gene leads to reduced susceptibility of mice to mouse hepatitis virus infection. J Virol 75:8173-8186.
34. Erlich, J., Parry, G. C., Feams, C., Muller, M., Carmeliet, P., Luther, T., and Mackman, N. 1999. Tissue factor is required for uterine hemostasis and maintenance of the placental labyrinth during gestation. Proc Natl Acad Sci USA 96:8138-8143.
35. Iwaki, T., Sandoval-Cooper, M. J., Paiva, M., Kobayashi, T., Ploplis, V. A., and Castellino, F. J. 2002. Fibrinogen stabilizes placental-maternal attachment during embryonic development in the mouse. Am J Pathol 160:1021-1034.
36. Isermann, B., Hendrickson, S. B., Hutley, K., Wing, M., and Weiler, H. 2001. Tissue-restricted expression of thrombomodulin in the placenta rescues thrombomodulin-deficient mice from early lethality and reveals a secondary developmental block. Development 128:827-838.
37. Sun, W. Y., Witte, D. P., Degen, J. L., Colbert, M. C., Burkart, M. C., Holmback, K., Xiao, Q., Bugge, T. H., and Degen, S. J. 1998. Prothrombin deficiency results in embryonic and neonatal lethality in mice. Proc Natl Acad Sci USA 95:7597-7602.
38. Cui, J., O'Shea, K. S., Purkayastha, A., Saunders, T. L., and Ginsburg, D. 1996. Fatal haemorrhage and incomplete block to embryogenesis in mice lacking coagulation factor V. Nature 384:66-68.
39. Bugge, T. H., Xiao, Q., Kombrinck, K. W., Flick, M. J., Holmback, K., Danton, M. J., Colbert, M. C., Witte, D. P., Fujikawa, K., Davie, E. W., et al. 1996. Fatal embryonic bleeding events in mice lacking tissue factor, the cell-associated initiator of blood coagulation. Proc Natl Acad Sci USA 93:6258-6263.
40. Ning, Q., Liu, M., Kongkham, P., Lai, M. M., Marsden, P. A., Tseng, J., Pereira, B., Belyavskyi, M., Leibowitz, J., Phillips, M. J., et al. 1999. The nucleocapsid protein of murine hepatitis virus type 3 induces transcription of the novel fgl2 prothrombinase gene. J Biol Chem 274:9930-9936.
41. Poutanen, S. M., Low, D. E., Henry, B., Finkelstein, S., Rose, D., Green, K., Tellier, R., Draker, R., Adachi, D., Ayers, M., et al. 2003. Identification of Severe Acute Respiratory Syndrome in Canada. N Engl J Med. epub Mar. 31, 2003.

1. A method for treating a subject afflicted with chronic viral hepatitis B, comprising administering to said subject an inhibitor of Fgl2 in an amount effective to reduce Fgl2-mediated fibrin deposition.

2. A method according to claim 1, wherein the inhibitor of Fgl2 is an antibody that binds to Fgl2.

3. A method according to claim 2, wherein the Fgl2 antibody is a monoclonal antibody.

4. A method according to claim 1, wherein the subject is afflicted with marked chronic viral hepatitis B.

5. A method according to claim 4, wherein the inhibitor of Fgl2 is an antibody that binds to Fgl2.

6. A method according to claim 5, wherein the Fgl2 antibody is a monoclonal antibody.

7. A method according to claim 1, wherein the subject is afflicted with minimal chronic viral hepatitis B.

8. A method according to claim 7, wherein the inhibitor of Fgl2 is an antibody that binds to Fgl2.

9. A method according to claim 8, wherein the Fgl2 antibody is a monoclonal antibody.
